(19) United States
(12) Patent Application Publication
(10) Pub. No.: US 2020/0077706 A1
(43) Pub. Date: Mar. 12, 2020

(54) INTELLIGENT INHALANT DISPENSING SYSTEM AND APPARATUS

(71) Applicant: Loop Laboratories, LLC, Chicago, IL (US)

(72) Inventors: Scott H. Wilson, Chicago, IL (US); Greg Ettenson, Austin, TX (US); Matteo Iavicoli, Chicago, IL (US)

(21) Appl. No.: 16/563,623

(22) Filed: Sep. 6, 2019

Related U.S. Application Data
(60) Provisional application No. 62/810,006, filed on Feb. 25, 2019.
(60) Provisional application No. 62/728,766, filed on Sep. 8, 2018.

(51) Int. Cl. A24F 47/00; A61M 15/06; G16H 20/10; A61M 15/00

(52) U.S. Cl. CPC A24F 47/008; A61M 15/06; A61M 2205/3584; A61M 15/0065; A61M 2205/8206; G16H 20/10

(57) ABSTRACT

An intelligent inhalant dispensing system comprising a smart substance delivery component configured to provide information identifying the specific substance stored therein for inhaling, and a smart inhalation device configured to receive the substance identifying information from the substance delivery component and adjust one or more settings of the inhalation device based thereon. The adjustable settings of the inhalation device can include heating temperature and duration, and/or dosage amount, and each substance may have different recommended values for these settings. The system can further customize the adjustable settings of the inhalation device based on user-specific settings, such as user profile information. The inhalation device can also be configured to track, monitor, and control usage of the cartridge.

### CROSS-REFERENCE TO RELATED APPLICATIONS

This application claims the benefit of U.S. Provisional Patent Application No. 62/728,766, filed on Sep. 8, 2018, and U.S. Provisional Patent Application No. 62/810,006, filed on Feb. 25, 2019, both of which are incorporated herein by reference in their entirety.

### TECHNICAL FIELD

This disclosure relates to inhalation devices, including vaporizers and inhalers, and more specifically to systems and apparatus for controlling and monitoring the administration of an inhalant using such devices.

### BACKGROUND

There are various forms of inhalation devices for converting a medicine or other substance into a vapor, aerosol, mist, or other inhalable form, and delivering the substance to the lungs for inhalation. One type of device is the vaporizer, or vape, which electrically heats a liquid substance into a vapor upon activation and delivers the vapor through a mouthpiece for user inhalation. For example, a typical vaporizer may include (1) a storage compartment for holding a solution or substance to be vaporized, (2) an atomizer or other heating element for vaporizing the substance, (3) a power source, such as, for example, a rechargeable lithium-ion battery, for powering the atomizer and/or delivery system, and (4) a mouthpiece to deliver the vapor for user inhalation. When a button is pressed, the heating element fires or activates to atomize or vaporize the solution as it is drawn across the heating element. Suction pressure is simultaneously supplied by the user at the mouthpiece, first pulling the solution across the heating element and then pulling the vaporized liquid through a barrel or delivery tube and out through the mouthpiece.

Another type of inhalation device is the inhaler, or puffer, which has traditionally been used for delivering medication into the body via the lungs, mainly for the treatment of asthma and chronic obstructive pulmonary disease. Inhalers deliver a fixed or metered dose of medication, or other substance, in aerosol form upon activation. As an example, a typical inhaler may include a solution in a pressurized canister, a dispensing actuator, a metering valve that controls the amount of solution released when activated, and a mouthpiece for delivering the aerosolized solution to the user. One form of inhaler is a nebulizer, which breaks up a liquid solution into a mist as it is being dispensed. This may be done in various ways, such as, e.g., by using ultrasonic waves, oxygen, or compressed air.

With the recent growth in vaping and the legalization of certain cannabis-based products, the demand for personal inhalation devices that can deliver cannabis and other plant-based substances or herbs has risen dramatically, for both medicinal and non-medical purposes. Such devices are typically portable, self-contained devices that come in varying sizes and/or forms, including, for example, slim cylindrical “vape pens” designed to look and feel like a traditional pen, vaporizers with elongated rectangular housings designed to fit easily within a pocket or wide rectangular housings designed to fit comfortably within a hand, inhalant devices that look and operate like traditional, medicinal inhalers, dab devices designed for use with oil or wax cartridges, as well as many others.

Personal inhalation devices come in at least two varieties: wholly disposable or at least partially reusable. Among the reusable devices, some have replaceable substance delivery components (such as, e.g., cartridges, pods, canisters, etc.) that can be discarded once depleted and replaced, for example, with a new, pre-filled component. Other reusable devices have refillable substance delivery components that can be refilled by the user or by an appropriate dispensary. Reusable inhalation devices have the added advantage of allowing the user to change the makeup or composition of the substance being inhaled. For example, cannabis-based products may come in different blends or strains, and a user may want different products at different times or for different needs (e.g., pain relief, sleep-inducement, relaxation, etc.).

However, many existing cartridges, pods, canisters, and other substance delivery components do not contain labels or information specifying which chemicals or substances are stored therein and/or instructions for preparing (e.g., heating) or otherwise using the substances. For example, some strains of cannabis may require a different heating profile or dosage amount than others. The dispensary or other merchant that sells the component to the user may provide this information verbally or in print, but many users forget or misplace this information fairly easily. Another drawback of conventional inhalation devices is that most devices do not keep track of the substances that were previously delivered using that device. As a result, when the same inhalation device is reused with different cartridges, for example, or the same cartridge is reused for different types of substances, residue or build up from previous uses may get mixed in with the substance contained in the currently-loaded cartridge, thus creating unplanned combinations with potentially unwelcome effects.

Still another drawback of existing inhalation devices is the lack of accurate dosing or metering of the substance to be inhaled. As an example, the amount of solution released with each use can be imprecise when using certain inhalation devices. For some existing vaporizers, the amount of solution released will depend on how long the user intakes or pulls in air. Also, most existing inhalation devices will dispense each time there is actuation of the device, with no measurements or controls in place to track or limit, for example, the number and/or frequency of uses, or monitor the amount of solution being dispensed. These drawbacks present a serious chance for abuse of the inhalant, particularly as inhalation devices are used more frequently for non-medicinal purposes.

### SUMMARY

Embodiments include an intelligent inhalant dispensing system comprising at least one “smart” cartridge configured to provide information about the contents of the cartridge and a “smart” inhalation device configured to obtain information from a smart cartridge installed therein and to facilitate monitoring, measuring, controlling, and/or tracking usage of the cartridge or device. Each smart cartridge may contain information identifying the substance contained in the cartridge, and in some cases, dosing and/or heating profile information specific to the substance. The smart inhalation device may contain information specific to the user of the device, and may combine this information with the information from the cartridge to make dosing and administration decisions. For example, the inhalation device may work in association with the smart cartridge to control dosing of the substance contained in the cartridge, monitor the user's inhalant consumption over time, provide the user with various information about the substance, such as, e.g., flavor profile, common side effects, heating profile, etc., and provide recommendations for new products or substances that the user may enjoy based on the user's consumption history.

Embodiments of the smart inhalation device can include various use-related features, such as, for example, a dosage administration system configured to electronically monitor dosing and prevent excessive dosing, and a cartridge identification system configured to identify the cartridge being used and provide dosage and/or product preparation information associated therewith. Some embodiments include a preparation system configured to monitor preparation of the substance to be inhaled (e.g., through shaking, heating temperature and/or time, etc.) and provide an indication when the substance is ready for use. One or more features may require the use of wireless communication with a personal electronic device, such as, e.g., a smartphone.

One exemplary embodiment includes an intelligent inhalant system comprised of an inhalant dispensing device designed to receive a smartpod canister that houses and is able to read and diagnose a disposable smartpod cartridge containing a solution to be dispensed. Each cartridge contains specific information about the solution content, and may also contain dosing information that may be specific to the solution. The canister may contain information specific to the user of the system, and combines this information with the information from the cartridge to make dosing and administration decisions. The dispensing device works in association with the smart pod cartridge to control dosing of the solution, provide the user with various information about the solution, and monitor the user's inhalant consumption over time.

Another exemplary embodiment includes an inhalation device comprising an inhalant delivery mechanism configured to deliver a substance for user inhalation; a communications device configured to receive data comprising substance-identifying information; and a dosing module configured to control one or more settings of the inhalant delivery mechanism using product preparation information associated with the substance identified by the received data.

Yet another exemplary embodiment includes an inhalant dispensing system comprising a substance delivery component configured to store a substance, and comprising a data module configured to store data associated with the substance; and an inhalation device comprising an opening for receiving the substance delivery component, an inhalant delivery mechanism configured to deliver the substance to a user for inhalation, and an electronics module configured to receive the data from the data module and, based on the received data, control one or more settings of the inhalant delivery mechanism.

Still another exemplary embodiment includes a method of customizing an inhalant dispensing experience using an inhalation device comprising at least one processor, a communications device, and at least one outlet for dispensing a substance to a user in inhalable form. The method comprises receiving, via the communications device, substance-identifying information for a substance; determining, using the at least one processor, product preparation information associated with the substance-identifying information; configuring, based on the product preparation information, one or more settings for controlling delivery of an inhalable form of the substance, using the at least one processor; and in response to receiving a user input, providing the substance for user inhalation via the at least one outlet.

While certain features and embodiments are referenced above, these and other features and embodiments will be, or will become, apparent to one having ordinary skill in the art upon examination of the following figures and detailed description. It is intended that all such additional embodiments and features included within this description, be within the scope of the invention, and be protected by the accompanying claims.

### BRIEF DESCRIPTION OF THE DRAWINGS

For a better understanding of the invention, reference may be made to embodiments shown in the drawings identified below. The components in the drawings are not necessarily to scale and related elements may be omitted, or in some instances proportions may have been exaggerated, so as to emphasize and clearly illustrate the novel features described herein. In addition, system components can be variously arranged, as known in the art. Further, in the drawings, like or substantially similar elements may be labeled with the same reference numerals. However, sometimes these elements may be labeled with differing numbers, such as, for example, in cases where such labeling facilitates a more clear description. Such labeling and drawing practices do not necessarily implicate an underlying substantive purpose. As stated herein, the specification is intended to be taken as a whole and interpreted in accordance with the principles of the invention as taught herein and understood to one of ordinary skill in the art.

FIG. 1 is a prospective view of an exemplary smart cartridge in accordance with certain embodiments.

FIG. 2 is a section view of the smart cartridge shown in FIG. 1 in accordance with certain embodiments, where the section is taken along the length of the cartridge.

FIG. 3 is an exploded view of an exemplary smart canister being loaded with the smart cartridge of FIG. 1, in accordance with certain embodiments.

FIG. 4 is a perspective view along the length of the smart canister of FIG. 3 with the smart cartridge of FIG. 1 installed therein, in accordance with certain embodiments.

FIG. 5 is an exploded view of an exemplary intelligent inhalant dispensing system showing the smart canister of FIG. 4 being loaded into an exemplary inhalation device, in accordance with certain embodiments.

FIG. 6 is a perspective view along the length of the inhalation device of FIG. 5 showing the smart canister of FIG. 4 installed therein, in accordance with certain embodiments.

FIG. 7 is an exploded, rear perspective view of another exemplary intelligent inhalant dispensing system showing an exemplary smart substance delivery component being loaded into an exemplary smart inhalation device, in accordance with certain embodiments.

FIG. 8 is a block diagram of an exemplary intelligent inhalant dispensing system, in accordance with certain embodiments.

### DETAILED DESCRIPTION

The description that follows describes, illustrates and exemplifies one or more particular embodiments of the invention in accordance with its principles. This description is not provided to limit the invention to the embodiments described herein, but rather to explain and teach the principles of the invention in such a way to enable one of ordinary skill in the art to understand these principles and, with that understanding, be able to apply them to practice not only the embodiments described herein, but also other embodiments that may come to mind in accordance with these principles. The scope of the invention is intended to cover all such embodiments that may fall within the scope of the appended claims, either literally or under the doctrine of equivalents.

In this application, the use of the disjunctive is intended to include the conjunctive. The use of definite or indefinite articles is not intended to indicate cardinality. In particular, a reference to “the” object or “a” and “an” object is intended to denote also one of a possible plurality of such object

In the following description, elements, circuits and functions may be shown in block diagram form in order to not obscure the present disclosure in unnecessary detail. Additionally, block definitions and partitioning of logic between various blocks is exemplary of a specific embodiment. Further, those of ordinary skill in the art will understand that information and signals as depicted in the block diagrams may be represented using any variety of different technologies or techniques. For example, data, instructions, signals or commends may be represented in the figures, and which also would be understood as representing voltages, currents, electromagnetic waves or magnetic or optical fields, or combinations thereof. Additionally, some drawings may represent signals as a single signal for clarity of the description; and persons skilled in the art would recognize that the signal may represent a bus of signals. Various illustrative logic blocks, modules and circuits described in connection with embodiments disclosed herein may be implemented or performed with one or more processors. As would be appreciated and understood by persons of ordinary skill in the art, disclosure of separate processors in block diagrams may indicate a plurality of processors performing the functions or logic sequence disclosed herein, or may represent multiple functions or sequence performed on a single processor.

The term “substance delivery component” is used herein to denote any type of device that includes a storage compartment for holding a substance to be inhaled and is configured for coupling to an inhalation device. Exemplary substance delivery components include cartridges (e.g., dab or wax cartridges, oil cartridges, etc.), pods (e.g., dab or wax pods, oil pods, dry herb pods, etc.), canisters (e.g., inhaler canisters, canisters configured to hold a cartridge, etc.), and the like. The substance stored in the substance delivery component can also vary and may include liquids, oils, concentrates, waxes, dry herbs, and other plant-based products. In some instances, the term “cartridge” is used for the sake of brevity but should be understood as including all types of substance delivery components. In some cases, the substance delivery component also includes a heating element for heating the substance into a vapor, and a mouthpiece to facilitate user inhalation, for example, as found in conventional cartridges and pods.

The term “inhalation device” is used herein to denote any type of personal inhalant dispensing device that is configured to receive a substance delivery component, prepare an inhalable form of the substance contained in the substance delivery component, and dispense the prepared substance for user inhalation. In some cases, the inhalation device also provides power to the substance delivery component, for example, where the substance delivery component includes a substance that requires heat in order to be vaporized, or otherwise requires power to prepare the substance for inhalation. In such cases, the inhalation device can include a rechargeable or disposable battery. The inhalation device can also include a coupling device for securing the substance delivery component therein. Exemplary inhalation devices include vape pens and other vaporizers for converting a substance into a vaporized form, inhalers or puffers for converting a substance into an aerosolized form, devices configured for dabbing use, and other personal inhalant dispensers.

The term “substance” is used herein to denote any type of product capable of being inhaled by a user, for example, upon conversion to a vapor, aerosol, mist, or other inhalant form, and is not limited to a particular consistency or format. For example, substance may include various types of cannabis, tobacco, and other plant-based products, including blends and/or strains thereof, and may come in various forms, including, for example, a wax or other concentrate, an oil or other liquid solution, a dry herb, flower, or other solid material, and others. Substance may also include medicinal products.

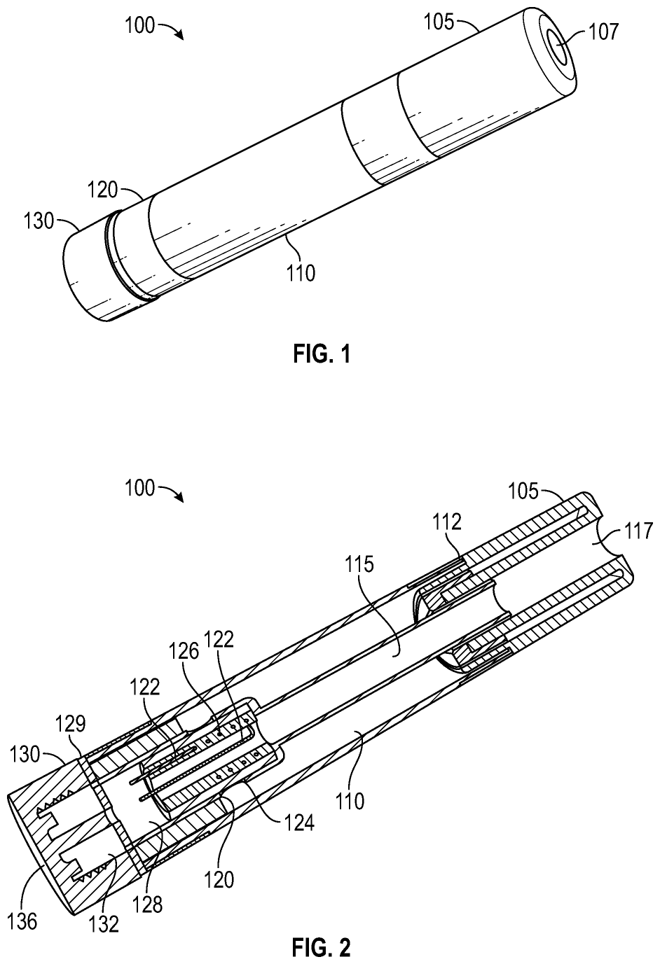

FIG. 1 illustrates an exemplary smartpod cartridge 100 (also referred to herein as a “smart cartridge”). The terms “smartpod” and “smart cartridge” are used to denote that the cartridge, or other substance delivery component, has data available for interpretation, and in some cases, at least a memory capability. Though other shapes could be employed, cartridge 100 is cylindrical in shape, having an opening 107 for dispensing an aerosol, vapor, or other inhalant at a first end, and a data cap 130 at the opposing end. The majority of the length between the opening 107 and the data cap 130 comprises a solution compartment 110 where solution is housed before being converted to an inhalable form for delivery to the user. This conversion function is performed by converter 120, which may be an atomizer, a heating element, or other appropriate product preparation device. The opening 107 is formed in a top cap 105 that is removable for purposes of filling the solution compartment 110 during manufacture.

FIG. 2 provides a section view of the smartpod cartridge 100, taken down its longitudinal centerline to illustrate various components of an inhalant delivery mechanism provided inside the cartridge 100. As shown, the opening 107 extends into the cartridge 100 to connect with a dispensing tube 115 that leads through the center of the solution compartment 110 back to the converter 120. The pre-converted solution is stored in the area around the dispensing tube 115, and is maintained within the cartridge by the top cap 105, which is pressed into a fill opening 112 of the solution compartment 110. The converter 120 not only serves to convert dosages of solution into aerosol or vapor for dispensing to the user, it also acts as a mechanical gateway or plug to contain the solution within the solution compartment 110. The converter 120 provides dispensing doors 124 along the outer walls of the converter 120 that selectively open to allow entry of solution into a heating chamber 126. When a user gives an indication that a dosage is requested (such as by pressing a button on inhalant device 300—See, e.g., FIG. 6), the doors 124 may open to allow a dosage of solution into the heating chamber 126, and a heating element within the converter 120 is temporarily engaged to convert the solution to an inhalable form, which is then drawn through delivery tubes 122 into an inhalant compartment 128 and, as desired, up through dispensing tube 115 and out the opening 107. Other known forms of conversion from solution to mist or other inhalable form using an inhalant delivery mechanism could be employed, such as an atomizer, or a vibration or ultrasonic wave generator.

A wall 129 forms the far end of the inhalant compartment 128, and separates the solution to be converted from the data cap 130. The data cap 130 is a removable cap that is twisted onto one or more threaded extensions 132 protruding from the wall 129. The data cap 130 is factory installed, and houses information about the solution contained within the smartpod cartridge. The data cap 130 is in electronic communication with the converter 120, and controls the opening of dispensing doors 124, operation of the heating element or other product preparation device, or both, and is thus able to control whether and how much solution is dispensed. It also records information about any dosage provided into a memory chip (not shown), such as the amount of solution dispensed and the time it was dispensed. This information is then communicated via an identification module 136, which may be a passive RFID tag or similar wireless communication protocol readable by a reader in the smartpod canister 200, as discussed below.

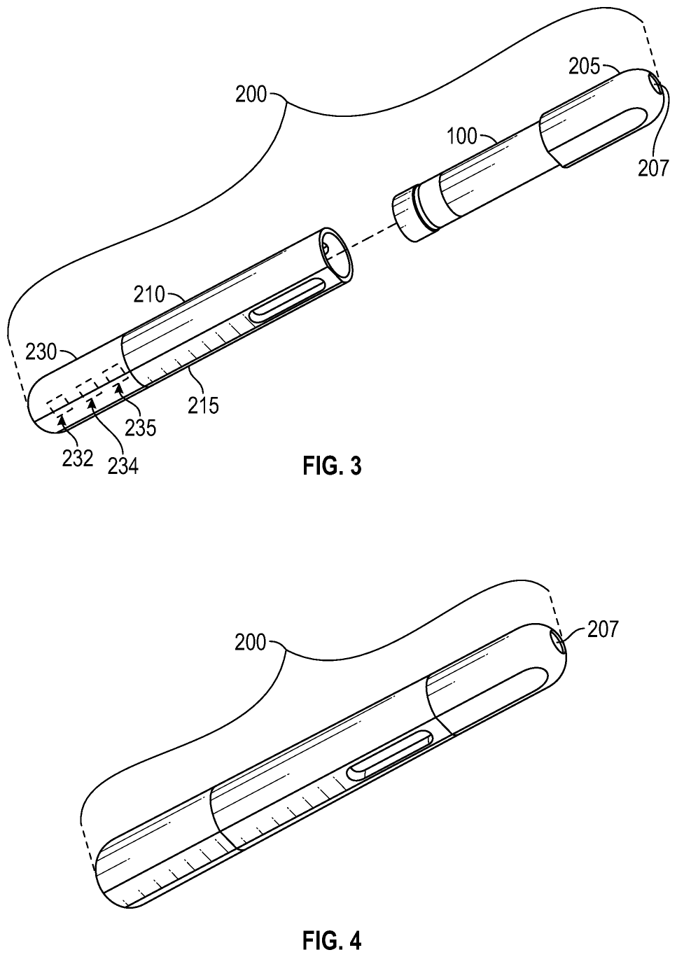

FIG. 3 illustrates a smartpod canister 200 (also referred to herein as a “smart canister”) that is used to house a smartpod cartridge 100. While the cartridges are designed to be disposable once the solution has been dispensed, the canisters are reusable, and may be loaded with information specific to the user or owner of the canister 200. The canister 200 may also be in wireless communication with an software application that presents a user interface to the user, such as, e.g., on a smartphone, tablet, or other computer device, or to a user account accessible via the internet, such that the user (or medical professionals, etc.) may interface with logic contained within canister 200 to tailor dosages and provide for permissions on dispensing aerosol solution to the user.

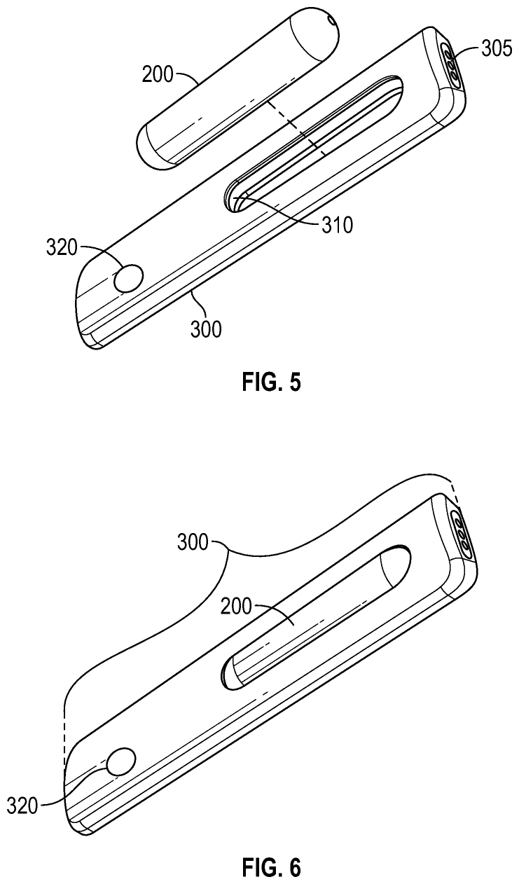

As shown in FIG. 3, the smartpod canister 200 shown in the featured embodiment is longitudinal in shape and is comprised of a base 210 and a cap 205. The cap 205 is designed to fit reasonably snugly over cap 105, such that the cartridge will not fall out of cap 205 even if suspended therefrom without the base 210 installed. Base 210 is then pressed over the exposed portion of the canister 100, and fitted up against cap 205. When installed, base 210 and cap 205 form an enclosed housing around a cartridge 100, such as shown in FIG. 4. An observation window 215 is provided along a length of the base 210 that aligns with a length of the solution compartment 110 when a cartridge 100 is properly loaded in the canister 200. Provided that the outer walls of the solution compartment 110 are transparent, the observation window 215 provides a user with a visual indication of the amount of solution remaining in the cartridge 100.

At the proximal end of the canister 200 is an opening 207 in the cap 205 that is in communication with the opening 105 at the end of the cartridge 100 and forms a further extension of dispensing tube 115 through which aerosol is drawn when being dispensed. At the distal end of the canister 200 is a communications module 230, which comprises a reader 232 to read information from the cartridge 100, a memory 234 to store information specific to the user, and a processor 235 to process dosage instructions and other data, and to provide instructions to the data cap 130 as to any controls that should be placed on amounts to be dispensed of the solution within the currently installed cartridge 100.

Once loaded with a cartridge 100, the canister 200 is itself then installed in an inhalation device such as inhalant device 300. The inhalant device 300 can take many forms, and may primarily provide a comfortable holding interface for the user. As shown in FIG. 5, the inhalant device 300 of the illustrated embodiment provides a receiving slot 310 into which the canister 200 is pressed. FIG. 6 shows a canister 200 properly loaded into the receiving slot 310. The receiving slot 310 has an open passageway to dispensing outlets 305 at a first end of the inhalant device 300. During use, this first end (and the corresponding dispensing outlets 305) is placed into a user's mouth. The user then presses an actuator 320 that triggers the heating element of the cartridge 100, and draws the inhalant through the dispensing tubes 305 by inhaling. Of course whether and how much inhalant is dispensed depends on controls within the overall dispensing system as described above.

The inhalant dispensing system herein described can be employed for a variety of practical uses. For example, the system can track a user's consumption of certain solutions and prevent the user from overdosing or taking in more solution than is proscribed over a period of time. Contrarily, if a certain dosage is required to be administered, the system can send an alert that it is time to take a dosage, or that the user has fallen behind in taking dosages. This alert could be sent, for example, to a user's smart watch or smartphone via Bluetooth, Near Field Communication (NFC), or other short-range wireless communication protocol. The information could also be stored and accessible by a reader used by medical professionals, or uploaded to the user's medical charts so that a medical professional can monitor dosaging and make adjustments remotely as necessary to the amount to be dispensed.

The system can also be used to confirm the contents of a canister or cartridge, and report this to the user before the user inhales the solution. Content information and other data, such as the manufacturer, the location and date of solution fill, etc., can be stored on the RFID within the data cap 130 of the canister and thereby made available to the user. When presented, the data could be combined with information in the memory 234 of the canister and processed to provide warnings or alerts to the user via an application user interface on the user's smartphone, etc. These warnings could be simply based off information within the data cap RFID (the solution has expired, the solution contains carcinogens, etc.), or it may be combined with data specific to the user stored within memory 234 (the user has had bad reactions to this particular solution in the past, the user is allergic to a substance within this solution, or the user has marked and identified via the user interface that this is a solution that makes her or him drowsy, etc.).

Other optional features include indicative lighting and a user identification system. For example, lighting along a panel of the inhalant device 300 (or the exposed surface of the canister 200) could be used to indicate an alert or dispensing problem to the user in an instant fashion so that consultation with a separate user interface is not required. For example, a yellow light could indicate that a full dosage has been restricted for some reason, a red light could indicate a dosage is not presently available, and a green light indicates a full dosage has been dispensed. More information as to why a red or yellow light shows up could then be accessed via the user interface. A user identification system could be employed such as through use of a fingerprint reader on the inhalant device. In this manner, the system could confirm the user and access the appropriate user information stored within memory 234.

The cartridge could also contain information about the solution's form, viscosity and blend of materials such as cannabis extracts, terpenes, nutraceuticals, essential oils, or cannabinoids, as well as information on appropriate usage, effectiveness, side effects, etc. The processor could use this information to adjust its heating temperature, length of heating time, and other dispensing characteristics to maximize effectiveness of the solution or obtain a desired effectiveness. The system could also convey information to the user about number of dosages left, and could automatically place an order for a new cartridge when dosage is running low.

Thus, an inhalant dispensing system for controlling dosages is described herein. The system comprises a disposable cartridge at least partially filled with a solution to be dispensed, the cartridge configured to selectively allow entry of solution into a conversion chamber that converts it to an aerosol mist for disbursement to a user; a data cap connected to the cartridge and forming a portion thereof, the data cap housing an identification module containing information about the solution within the cartridge; a canister for housing the cartridge; and an inhalant device comprising one or more dispensing outlets in communication with a receiving slot configured to receive and hold the canister, the dispensing outlets aligning with an opening in the canister and an opening in the cartridge to provide an open pathway to an outlet of the conversion chamber. Moreover, the canister comprises a reader in wireless communication with the identification module to collect information about the solution, a memory for storing information specific to a user of the dispensing system, and a processor in communication with the memory and reader to process dispensing determinations and messages to the user.

While FIGS. 1-6 illustrate one exemplary form of an intelligent inhalant dispensing system, it should be appreciated that other forms of substance delivery components and/or inhalation devices may be used to intelligently deliver a substance to a user for inhalation in accordance with the techniques described herein. For example, embodiments may include other types of vaporizers and/or cartridges. Some embodiments may include a smart cartridge that is directly inserted into the vaporizer, i.e. without first being inserted into a canister. In such cases, the smart cartridge may communicate data to the vaporizer. Some embodiments may include a vaporizer comprising multiple cartridges, each cartridge storing a different substance (e.g., a different strain, blend, or material). The multi-cartridge vaporizer may include an electronics system configured to control and monitor usage of the cartridges, including accurate dosing and tracking of doses. In addition, the electronics system may be communicatively coupled to a remote device (e.g., smartphone) of the user to facilitate these and other features of the vaporizer. Applying the techniques described herein, each cartridge of the multi-cartridge vaporizer may be configured as a smart cartridge configured to store data about the particular substance included therein, and the vaporizer can be a smart inhalation device configured to obtain the data from the smart cartridges installed therein and control dosage, vaporization, and/or other usage features based on the specific substance information received from the cartridges.

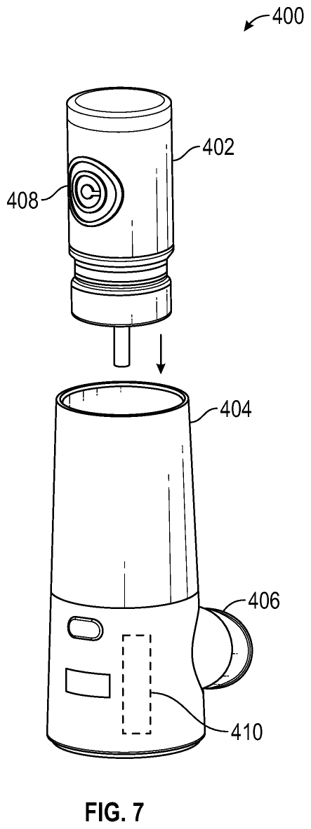

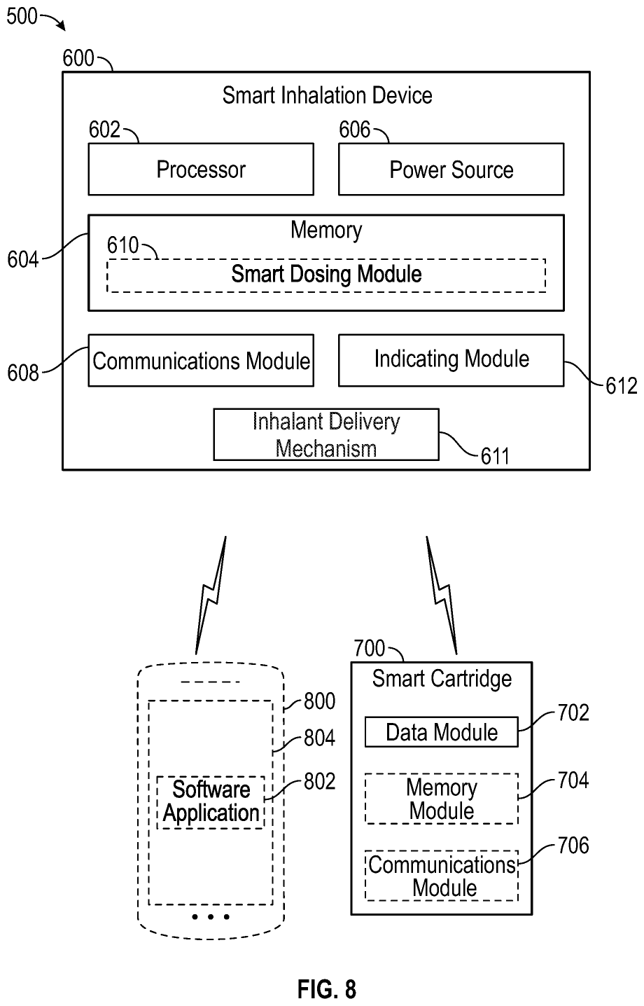

As another example, some embodiments may include an inhaler-type inhalation system. FIG. 7 depicts an exemplary intelligent inhalant dispensing system 400 comprised of a smart canister 402, or substance delivery component, containing a substance to be aerosolized and a smart inhaler 404 configured to receive the canister 402 through an open top and dispense the aerosolized substance through a mouthpiece 406. The canister 402 includes a smart label 408 comprising data related to the substance residing inside the canister 402, similar to the data stored in the data cap 130 of FIG. 1. In embodiments, the smart label 408 may be an adhesive sticker or decal attached to an outer surface of the canister 402 and comprising a radio frequency identification (RFID) tag or other short-range device. The inhaler 404 includes an electronics module 410 within a lower chamber of the inhaler 404 that is configured to enable monitoring, tracking, dosing, and/or communication features of the inhaler 404, and includes a corresponding RFID reader or other communication device for reading data from the canister's smart label 408 and implementing dosing controls based thereon, for example. In embodiments, to carry out these and other features, the electronics module 410 may include a processor, memory, power source, communications module, and/or indicating module, for example, like the smart inhalation device 800 shown in FIG. 8 and described herein. According to embodiments, the inhaler 404 may be a metered-dose inhaler (MDI) that is configured to deliver a pre-determined amount of solution, or dose, to the lungs in the form of a short burst of aerosolized substance, or mist. In embodiments, the mouthpiece 406, a dispensing actuator for releasing the solution from the pressurized canister 402, a metering valve for controlling the amount of solution released when activated, an outlet of the canister 402 for dispensing the aerosolized solution into the mouthpiece 406, and/or other internal components of the cartridge 402 or inhaler 404 may collectively form an inhalant delivery mechanism of the inhalant system 400.

FIG. 8 depicts an exemplary intelligent inhalant dispensing system 500, in accordance with certain embodiments. The system 500 comprises a smart inhalation device 600 and a smart cartridge 700 capable of wirelessly communicating with the inhalation device 600 for tracking, monitoring, dosing, and/or controlling purposes. In embodiments, the cartridge 700 stores data that can be read by the inhalation device 600 and used to control various features of the device 600, including dosage control. The smart inhalation device 600 may be implemented using any of the inhalation devices described herein, including the inhalant device 300 shown in FIG. 3, the inhaler 404 shown in FIG. 7, and the multi-cartridge vaporizer, or other types of inhalant dispensing devices in accordance with the techniques described herein. Likewise, the smart cartridge 700 may be implemented using any of the cartridges described herein, including the smartpod cartridge 100 and/or canister 200 shown in FIG. 3 and the smart cartridge 402 shown in FIG. 7, or any other type of substance delivery component in accordance with the techniques described herein.

The smart cartridge 700 includes a data module 702 for storing data related to the substance residing inside a storage compartment (not shown) of the cartridge 700. The data may include substance identifying information that identifies the exact organic material residing in the cartridge 700 and a profile or content of the material. For example, the substance identifying information may include specific strain, formulation, and/or blend information, including CBD levels, THC levels, strain name, potency, terpene profile, cannabinoid profile, and/or ratio of psychoactive vs. non-psychoactive chemicals. In some cases, the substance identifying information may be pre-loaded onto the data module 702 by the original manufacturer in a product filling line (e.g., in the case of disposable cartridges). In other cases, the substance identifying information may be added or uploaded to the data module 702 at the time of filling or re-filling the cartridge 700 with a substance by the user or dispensary that performs the filling.

In some embodiments, the data module 702 also stores product preparation information specific to the substance stored in the cartridge, such as, e.g., mixing or shaking requirements, heating temperature, heating time, etc. In other embodiments, this product preparation information is retrieved by the inhalation device 600 from another location or device in response to receiving the substance identifying information from the cartridge 700.

The data module 702 can include an RFID tag, a near-field communication (NFC) chip, a QR code, a barcode, or other identification device capable of storing the substance identifying information and being read by, or otherwise providing this information to, the smart inhalation device 600. In some cases, the data module 702 is embedded in or included on a printed label (e.g., adhesive sticker or decal) or other object that is attached to an outer surface of the cartridge 700 (such as, e.g., smart label 408 shown in FIG. 7). In other cases, the data module 702 may be etched onto or otherwise embedded into the cartridge 700 itself, for example, in the case of a QR code or barcode. In still other cases, the data module 702 may be included in a separate component that is attached to the cartridge 700, for example, like the data cap 130 shown in FIG. 1.

In some embodiments, smart cartridge 700 includes a memory module 704 for storing the substance identifying information and/or other data, and a communications module 706 for providing the stored data to the inhalation device 600 and/or otherwise communicating with the device 600. The communications module 706 may be configured to transmit data from the memory module 704 to the inhalation device 600 using one or more wired or wireless technologies. In some cases, the communications module 706 may also be configured to receive data for storing in the memory module 704. The communications module 706 may comprise one or more transceivers, ports, modems, or other communications devices for facilitating communications using Bluetooth, NFC, Wi-Fi, or other wireless communications technology. In some cases, the smart cartridge 700 also includes a processor (e.g., microprocessor) to carry out one or more functions using software instructions stored in the memory module 704 and/or received via the communications module 706.

In some cases, the memory module 704 and communications module 706 are included in the smart cartridge 700 instead of the data module 702 for storing and communicating the substance identifying information. In other cases, the memory module 704 is configured to store data other than the substance identifying information stored in the data module 702, and the communications module 706 is for communicating only the data stored in the memory module 704, separate from the communication between the data module 702 and the inhalation device 600. As will be appreciated, other types of short-range wireless communication technology and/or data storage devices may be used to store substance information in the cartridge 700 and transfer the stored information to the inhalation device 600 or other component for identification, monitoring, tracking, and/or dosing purposes.

The memory module 704 can be configured to store usage data related to the smart cartridge 700, including number of doses left, number of doses used, initial number of doses, frequency of use, time of last use, time of each use, etc. In some cases, the usage data provided by the cartridge 700 may be used to help with ordering or re-ordering of the cartridge from the supplier once product levels are low. For example, usage data stored in the memory module 704 may be used by the supplier to discover which substances are being used more frequently and make recommendations for future orders based on use patterns. In some cases, the memory module 704 stores the product preparation information associated with the substance stored in the cartridge, such as, e.g., mixing or shaking requirements, heating temperature, heating time, etc.

As shown in FIG. 8, the smart inhalation device 600 includes at least one processor 602 and at least one memory 604 for storing software that may be executed by the processor 602 to implement one or more functions of the inhalation device 600. The processor 602 may communicate with the memory 604 and any other components of the inhalation device 600 using a data bus (not shown) or other appropriate medium. In embodiments, the processor 602 (e.g., data processor) can comprise one or more of a microprocessor, a microcontroller, a programmable logic array, an application-specific integrated circuit, a logic device, or other electronic device for processing, inputting, outputting, manipulating, storing, or retrieving data.

The inhalation device 600 also includes a power source 606 for powering the device 600. In some cases, the power source 606 is a rechargeable battery that also supplies power to the cartridge 700 once coupled to the inhalation device 600. In other cases, the power source 606 is separate from the battery for powering the cartridge 700.

The inhalation device 600 further includes a communications module 608. In embodiments, the communications module 608 comprises an appropriate data receiver configured to read or receive the substance identifying information and/or other data stored in the data module 702 of the cartridge 700. For example, the communications module 608 may include an RFID reader, NFC reader, QR code reader, or barcode reader depending on the type of identification device included in the data module 702. The communications module 608 may also be configured to facilitate wireless communications between the inhalation device 600 and an external or remote device, such as, e.g., a mobile device (e.g., smartphone, tablet, laptop, etc.) or other computing device of the user. In some cases, the communications module 608 may also be configured to facilitate wireless communications with the communications module 706 of the smart cartridge 700. The communications module 608 of the inhalation device 600 may comprise one or more transceivers, ports, modems, or other communications devices for facilitating such communications using Bluetooth, NFC, Wi-Fi, or other wireless communications technology.

The memory 604 (e.g., data storage device) can comprise one or more of electronic memory, nonvolatile random access memory (e.g., RAM), flip-flops, a computer-writable or computer-readable storage medium, a magnetic or optical data storage device, a magnetic or optical disc drive, a hard disk drive, or other electronic device for storing, retrieving, reading, or writing data. The memory 604 may store one or more software program modules or software instructions for execution by the processor 602. For example, the memory 604 may store a smart dosing module 610 comprising software instructions for retrieving substance identifying information from the cartridge 700 or otherwise identifying the cartridge 700, tracking usage of the cartridge 700 and/or the inhalation device 600, monitoring doses and controlling dosage during use of the inhalation device 600, controlling one or more settings of the cartridge 700, such as, e.g., heating temperature, heating time, and length of puff or other dosage amount, during use, and/or implementing customized settings received from a mobile device or a software application running thereon, using the techniques described herein.

In some embodiments, the memory 604 stores a program module or software instructions, as part of the smart dosing module 610 or separately, for automatically adjusting one or more pre-determined settings of the cartridge 700 and/or the inhalation device 600 based on the substance identifying information retrieved from the data module 702. In such cases, the software instructions include determining or obtaining product preparation information that is associated with the substance-identifying information. For example, the product preparation information can comprise a pre-determined or recommended value for each of the pre-determined settings that is tailored to the particular substance identified by the substance-identifying information.

According to embodiments, the product preparation information may be obtained by accessing a database that stores the product preparation information for each strain, blend, or other type of product on the market. For example, the database may be configured to store product preparation information in association with substance-identifying information for a plurality of different substances. In some cases, the database is stored in the memory 604 of the inhalation device 600. In other cases, the database may be stored in a cloud server (not shown) or other remote location and may be accessed by the inhalation device 600 (or smart dosing module 610) using the communications module 608 and the processor 602, in response to receiving substance identifying information from the smart cartridge 700.

In embodiments, the one or more pre-determined settings can control an inhalant delivery mechanism 611 included in the inhalation device 600. In some embodiments, the inhalant delivery mechanism 611 includes a heating element or other device for vaporizing a substance, such as the substance stored in the smart cartridge 700, or any other device used to prepare the substance for user inhalation (also referred to as “product preparation device”). In the case of a heating element, the pre-determined settings can include a temperature setting and a heat duration setting for the heating element, as described herein.

In other embodiments, the inhalant delivery mechanism 611 includes a power source configured to activate or provide power to a heating element included in the smart cartridge 700 (see, e.g., FIG. 2). For example, the smart cartridge 700 can be configured to electrically connect to the power source of the inhalant delivery mechanism 611 upon insertion of the cartridge 700 into an opening of the inhalation device 600 (e.g., receiving slot 310 shown in FIG. 5). Once connected, the heating element of the cartridge 700 may be activated, and may begin heating the substance stored in the cartridge 700, upon receiving power from the power source, and may continue to heat the substance so long as power is provided. In such cases, the pre-determined settings can include a power setting for controlling the power source of the inhalant delivery mechanism 611, as described herein. In some embodiments, the power source of the inhalant delivery mechanism 611 is the power source 606 shown in FIG. 8. In other embodiments, the inhalant delivery mechanism 611 has a separate power source, which may be, for example, a lithium ion battery, a rechargeable battery, etc.

In some embodiments, the inhalant delivery mechanism 611 also includes at least one outlet for dispensing the substance in inhalant form to the user, for example, as shown in FIGS. 2 and 7, and/or a mouthpiece for enabling user inhalation of the same. In such cases, the pre-determined settings can include a dosage amount for controlling how much substance is dispensed through the outlet, for example, during a single dose, as described herein.

The inhalant delivery mechanism 611 may also include other internal components of the inhalation device 600 that facilitate delivery of the inhalant, such as, e.g., dispensing doors 124 and/or delivery tubes 122 shown in FIG. 2, and can be electronically controlled by the smart dosing module, for example. As an example, in response to receiving a user input, e.g., via an actuation button or other user-interface of the inhalation device 600, the processor 602 may cause the inhalant delivery mechanism 611 to provide the substance in inhalable form to the user via the at least one outlet of the device 600. This may be achieved by causing the dispensing doors to open and stay open until a pre-determined amount of the substance is dispensed.

In some cases, the pre-determined settings can be material-specific settings, or device settings that need to be configured based on, or tailored to, the organic material included in the cartridge 700 and its profile. Such settings may be considered “universal” settings, for example, as opposed to user-customized settings. One such setting may be heat, or the temperature used to heat a given substance (e.g., using the heating element of the cartridge 700). For example, each substance may be associated with a specific temperature value, or range of values, that is recommended in order to avoid burning or undercooking the substance. Accordingly, the product preparation information for a given substance may include the recommended temperature value, or range of values. Another pre-determined setting may be duration, or the length of time a substance is heated. For example, an oil or other solution may not require as much time to heat up as a flower or dry herb. Accordingly, each substance may be associated with a specific heating time that is recommended to properly prepare the substance, and/or avoid burning or undercooking, and the product preparation information for a given substance may further include the recommended heat duration value.

In some cases, the temperature and heat duration settings may be controlled by directly configuring corresponding settings of the heating element based on the product preparation information. In other cases, the temperature and heat duration settings may be controlled by configuring a power output of the power source or battery that provides power to the heating element, such as, e.g., the power source 606, or otherwise activates the substance heating process. In such cases, the one or more pre-determined settings may include a power setting for controlling a power output of the power source 606, including, for example, the amount of power applied to the heating element by the power source 606 and the length of time the power source 606 is on, or otherwise capable of providing power to the heating element.

The pre-determined settings may also include a dosage setting for controlling the amount of substance that constitutes a single dose and/or is dispensed through the inhalant delivery mechanism 611 (e.g., via the one or more outlets included therein). In such cases, the product preparation information for a given substance can include a recommended dosage amount for that particular substance. In some cases, a single dose is characterized as a pre-determined amount or volume of the substance. In other cases, a single dose is measured by the length of the draw, or how long the user intakes or inhales from the device (e.g., 2 seconds or 2.5 seconds). The accurate dosage amount and type for a given substance may vary, depending on the type of strain or organic material and its profile (e.g., concentration, potency, chemical makeup, etc.). In some cases, the inhalation device 600 includes an indication system for indicating when a given dose is complete, for example, through vibrations generated by a haptic motor or light pulses emitted by an LED.

The pre-determined settings may include other settings of the inhalation system 500 that are configured based on the particular strain or substance residing in the cartridge 700. The recommended settings may be determined by the manufacturer, factory, dispensary, or other supplier of the substance.

In some cases, the one or more pre-determined settings can be further adjusted or configured based on user profile information for customizing or tailoring the settings according to a selected experience level. For example, the system 500 may have a plurality of preset profiles, including a “light” or “beginner” profile for setting the heating element to a lower temperature and heating the substance for a shorter heat time and/or taking a smaller dose, as compared to the recommended settings described above. The preset profiles may also include a “strong” or “advanced” profile for setting the heating element to a higher temperature and heating the substance for a longer heat time and/or taking a larger dose, compared to the recommended settings. The preset profiles may further include a recommended or regular setting for implementing the recommended heat, duration, and dose settings for that particular substance. The preset profile information may be pre-stored in the smart dosing module 610 or retrieved by the processor 602 via the communications module 608 from a remote database, like the other pre-determined settings.

As shown, the inhalation device 600 also includes an indicating module 612. The indicating module 612 may be configured to convey various types of information related to the inhalation device 600 to the user, such as, e.g., error messages related to operation of the cartridge, the battery, and/or other aspects of the device 600, a current operating mode (e.g., ready to use, in preparation, dose complete, etc.), whether a dose has been completed, low or depleted contents for a given cartridge, low battery power, and/or other notifications related to other detectable condition(s). In the case of multiple cartridges, the indicating module 612 may be configured to indicate or convey which cartridge(s) are currently selected, in use, and/or are available for use. The indicating module 612 can include one or more lights (e.g., light-emitting diodes (LEDs)), haptic devices (e.g., a vibrating motor or actuator), audio devices (e.g., a speaker or tweeter), and/or display devices (e.g., a touchscreen or LCD). In some embodiments, the indicating module 612 includes a haptic device configured to provide dynamic feedback to the user during use of the inhalation device 600. For example, the haptic device may be configured to vibrate when a single dose is complete and/or each time the user has completed a dose, where a dose comprises a pre-determined amount or content of substance and/or corresponds to a pull or inhale lasting a predetermined length of time (e.g., two seconds). The indicating module 612 may be in communication with the processor 602 and the memory 604, as well as other components of the device 600, in order to carry out its operations. For example, the indicating module 612 may be activated by the processor 602 upon receiving a signal indicating, or associated with, delivery of the pre-determined amount, or dose, of substance for user inhalation.

As shown in FIG. 8, the inhalation device 600 can be configured to wirelessly communicate with a mobile device 800 of the user. The mobile device 800 may be a smartphone, tablet, laptop, or other computing device capable of wirelessly communicating with the inhalation device 600. The mobile device 800 may include all or a portion of a software application 802 for controlling, monitoring, and/or tracking use of the inhalation device 600 and a display screen 804 (e.g., touchscreen and/or other display device) for displaying information generated by the software application 802 or otherwise related to the cartridge 700 and/or the inhalation device 600, and in some cases, for enabling user interaction with the software application 802.

Though not shown, the mobile device 800 may include at least one processor (e.g., microprocessor), memory element (e.g., electronic memory), and communications module (comprising, e.g., one or more wireless transceivers, modems, antennas, etc.) configured to implement various features of the software application 802. In particular, all or portions of the software application 802 may be stored in the memory, along with other data related to, or received from, the inhalation device 600. The processor may be configured to execute the software application 802 and present a user interface on the display screen 804. The user interface may be configured to enable user selection of certain settings for the inhalation device 600, such as, for example, the preset profiles described herein, and/or enter certain information, such as, e.g., genetic profile data, personal preferences of the user, user history data, etc. The communications module may be configured to communicate with the inhalation device 600 using, for example, Bluetooth, WiFi, or other wireless communication technology. In some embodiments, the mobile device 800 may use the communications module to access data and/or portions of the software application 802 that are stored remotely (e.g., in a cloud server).

The mobile application 802 can be configured to cooperate with the smart dosing module 610 of the inhalation device 600 in order to carry out one or more operations related to tracking, monitoring, and/or controlling use of the inhalation device 600 or the cartridge 700 installed therein. For example, in some embodiments, the smart dosing module 610 controls or adjusts one or more settings of the inhalation device 600 and/or the cartridge 700 (e.g., heat, duration, and dose) based on customized settings received from the mobile application 802. In some cases, the smart dosing module 610 provides the pre-determined material-specific settings to the software application 802, and the software application 802 further customizes or adjusts these settings based on user profile information provided by the user or otherwise obtained by the software application 802. In other cases, the software application 802, itself, identifies the pre-determined material-specific settings based on the substance identifying information obtained from the cartridge 700 by the inhalation device 600, and the smart dosing module 610 applies the settings received from the mobile device 800. In other embodiments, the processor 602 may use the smart dosing module 610 to automatically select and apply the pre-determined settings that are specific to the substance identified by the received data, until or unless the software application 802 provides customized user settings.

In some embodiments, the user profile information that may be used to customize settings for the inhalation device 600 can include genetic profile data obtained from a third-party, such as, e.g., 23andMe®. The genetic profile data may be used by the software application 802 to make personalized recommendations, such as, for example, which strains are best suited for the user, or is most likely to provide a desired effect, based on the user's genetic profile. In some embodiments, the user profile information may also include user preferences entered by the user, such as, e.g., preferred heating profile (e.g., temperature and heat time) for a specific strain, preferred dosage amount for a specific strain, preferred time of day (e.g., night or day) to use either a particular strain or any substance, desired effects when vaping (e.g., pain relief, relaxation, anxiety-relief, etc.), the user's experience-level to help select one of the preset profiles, and others. In some embodiments, the software application 802 and/or the smart dosing module 610 are configured to learn user preferences over time, including what the user likes and doesn't like, which dose of a given strain produces optimal effects for that user, etc., and store this learned information in the user profile information.

In some embodiments, the software application 802 can also cooperate with the smart dosing module 610 to track and monitor use of the inhalation device 600 and/or cartridge 700. For example, the smart dosing module 610 may record the number of puffs or doses taken during a given use or session, the time of day for each session, frequency of uses or amount of time between sessions, and/or other information related to tracking and monitoring use (also referred to herein as “usage data”). In some cases, the software application 802 and/or the smart dosing module 610 may control or prevent usage of the inhalation device 600 if the usage data exceeds or meets a predetermined threshold, such as, e.g., once a threshold number of doses have been reached for the day or within a set time period. The tracking information may also be used to recommend products to the user or remind the user when it's time to order another cartridge.

Thus, the techniques described herein can provide users with a customized inhalant dispensing experience that can be tailored to both the particular user and the particular substance to be inhaled by combining material-specific settings (e.g., heat, duration, dose, etc.), which are pre-determined or standardized for the substance contained in a given cartridge, with user-specific settings (e.g., recommended strains, genetic profile, user-entered preferences, learned preferences, user history, etc.), which are unique to each user. Embodiments include an intelligent inhalation system comprising embedded technologies that recognize a specific organic material when introduced into the system and based on that recognition, automatically adjust settings within the inhalation device to deliver an optimal experience based on the particular material's chemical profile. The system also provides accurate dosing capabilities for users. The intelligent inhalation system can be incorporated into various types of vaping and other inhalant dispensing technology and hardware including, but not limited to, oil cartridges, oil pods, ‘dab’ cartridges, ‘dab’ pods, and their associated battery/heater components. In addition, the system can be a mobile application-supported and -enabled experience. For example, users can view data, adjust settings, input feedback, and track consumption via their mobile device using a Bluetooth connection to the hardware component of the system, e.g., the inhalation device.

As an example, when a substance delivery component (e.g., cartridge or pod) is installed within its associated hardware component or inhalation device (e.g., a battery pen for the cartridge or battery pack for the pod), the device reads the ID device (e.g., QR code) of the cartridge or pod and based thereon, triggers software protocols to automatically adjust settings for that particular strain to deliver the best experience possible. The protocols can also ‘push’ recognized strain data to its associated mobile application for the user to view, track, adjust settings further, and input feedback. The mobile application may use software protocols, user generated input, and the genetic profiles of users, to generate personalized customization and controlled dosing. For example, the inhalation device can be pre-programmed with general settings optimized for each strain on the market. These pre-determined settings (e.g., heat, duration, dose, etc.) are solely based on the organic material and its profile. The mobile application may be configured to combine this plant data with the user's genetic profile and other personal information, thus resulting in a deeper level of customization, such as, e.g., recommendations for strains that are best suited for a user, or would produce user-desired effects. For example, the mobile application may be configured to know its user's specific genetic profile (if permitted by the user) and over time, learn what the user likes, what they don't like, and what dose for each strain is optimal.

The techniques described herein also provide an aerosol dispensing system for use in controlling and monitoring dosages of a solution administered via an inhaler. The system comprises a smartpod cartridge that has controls in place to limit the amount of solution converted to aerosol and dispensed, and also contains and provides information about the solution itself. The smartpod cartridge may be placed into a smartpod canister that reads information from the cartridge and processes it along with information specific to a particular user to make dosage determinations, provide warnings, and update dosage information for monitoring purposes. The smartpod canister can be removably placed within an inhalant device that provides an interface for the user to request dosages from the cartridge.

Moreover, the techniques described herein provide a method of customizing an inhalant dispensing experience using an inhalation device comprising at least one processor, a communications device, and at least one outlet for dispensing a substance to a user in an inhalable form. The method comprises receiving, via the communications device, substance-identifying information for a substance; determining, using the at least one processor, product preparation information associated with the substance-identifying information; configuring, based on the product preparation information, one or more settings of the inhalation device for controlling delivery of an inhalable form of the substance, using the at least one processor; and in response to receiving a user input, providing the substance for user inhalation via the at least one outlet.

The method can also comprise further adjusting at least one of the one or more settings based on user profile information for customizing the at least one setting, using the at least one processor. According to some aspects of the method, the one or more settings can comprise a temperature setting and a heat duration setting for controlling operation of a heating element configured to vaporize the substance. According to other aspects of the method, the one or more settings can comprise a dosage setting for controlling an amount of substance dispensed through the mouthpiece. According to still other aspects of the method, the one or more settings can comprise a power setting for controlling a power output of the power source. According to additional aspects of the method, the step of determining the product preparation information can comprise accessing, using the at least one processor, a database configured to store product preparation information in association with substance-identifying information for a plurality of different substances. In some cases, the method further comprises activating an indication device, using the at least one processor, in association with delivery of a pre-determined amount of the substance through the mouthpiece.

In certain embodiments, the process descriptions or blocks in the figures can represent modules, segments, or portions of code which include one or more executable instructions for implementing specific logical functions or steps in the process. Any alternate implementations are included within the scope of the embodiments described herein, in which functions may be executed out of order from that shown or discussed, including substantially concurrently or in reverse order, depending on the functionality involved, as would be understood by those having ordinary skill in the art.

It should be emphasized that the above-described embodiments, particularly, any “preferred” embodiments, are possible examples of implementations, merely set forth for a clear understanding of the principles of the invention. It will be understood by those skilled in the art that various changes may be made and equivalents may be substituted without departing from the scope of the novel and non-obvious techniques disclosed herein. Therefore, it is intended that the novel teachings of the invention not be limited to the particular embodiment(s) disclosed, but that they will include all embodiments falling within the scope of the appended claims.

What is claimed is:

1. An inhalation device, comprising:
an inhalant delivery mechanism configured to deliver a substance for user inhalation;
a communications device configured to receive data comprising substance-identifying information; and
a dosing module configured to control one or more settings of the inhalant delivery mechanism using product preparation information associated with the substance identified by the received data.

2. The inhalation device of claim 1, wherein the dosing module is configured to further adjust at least one of the one or more settings based on user profile information for customizing the at least one setting.

3. The inhalation device of claim 1, wherein the inhalant delivery mechanism comprises at least one outlet for dispensing the vaporized substance for user inhalation, and the one or more settings comprise a dosage setting for controlling an amount of substance dispensed through the at least one outlet.

4. The inhalation device of claim 1, wherein the inhalant delivery mechanism comprises a heating element operable to heat a substance into vapor, and the one or more settings comprise a temperature setting and a heat duration setting for controlling operation of the heating element.

5. The inhalation device of claim 1, wherein the inhalant delivery mechanism comprises a power source operable to provide power to a heating element for heating the substance into a vapor, and the one or more settings comprise a power setting for controlling a power output of the power source.

6. The inhalation device of claim 1, wherein the dosing module is further configured to: retrieve the product preparation information from a database for storing product preparation information in association with substance-identifying information for a plurality of different substances.

7. The inhalation device of claim 1, further comprising: a memory configured to store the dosing module.

8. The inhalation device of claim 1, further comprising: an indicating module configured to convey information associated with delivery of the substance to the user.

9. The inhalation device of claim 8, wherein the indicating module comprises a haptic device configured to vibrate in association with delivery of a pre-determined amount of the sub stance.

10. The inhalation device of claim 8, wherein the indicating module comprises a light device configured to provide a light-based notification during delivery of the substance.

11. The inhalation device of claim 1, wherein the communications device comprises a short-range wireless receiver.

12. An inhalant dispensing system, comprising:
a substance delivery component configured to store a substance, and comprising a data module configured to store data associated with the substance; and
an inhalation device comprising:
an opening configured to receive the substance delivery component,
an inhalant delivery mechanism configured to deliver the substance to a user for inhalation, and
an electronics module configured to receive the data from the data module and, based on the received data, control one or more settings of the inhalant delivery mechanism.

13. The inhalant dispensing system of claim 12, wherein the data comprises substance-identifying information for the substance stored in the substance delivery component.

14. The inhalation dispensing system of claim 13, wherein the electronics module controls the one or more settings by:
determining product preparation information associated with the substance-identifying information received from the data module, and
configuring the one or more settings of the inhalant delivery mechanism based on the product preparation information.

15. The inhalant dispensing system of claim 12, wherein the inhalant delivery mechanism comprises a heating element operable to heat the substance into a vapor, and the one or more settings control a temperature setting and a heat duration setting for the heating element.

16. The inhalant dispensing system of claim 12, wherein the substance delivery component further comprises a heating element operable to heat the substance into a vapor, the inhalant delivery mechanism comprises a power source configured to provide power to the heating element, and the one or more settings comprise a power setting for controlling a power output of the power source.

17. The inhalant dispensing system of claim 12, wherein the inhalant delivery mechanism comprises one or more outlets for dispensing the substance for user inhalation, and the one or more settings comprise a dosage setting for controlling an amount of substance dispensed through the one or more outlets.

18. The inhalant dispensing system of claim 12, wherein the data stored in the data module comprises usage data associated with the substance delivery component, and the electronics module is configured to prevent delivery of the substance for user inhalation if the usage data exceeds a predetermined threshold.

19. The inhalant dispensing system of claim 12, wherein the electronics module comprises a short-range wireless communications device operable to receive the data from the data module, and the data module comprises a short-range wireless communications device operable to transmit the data to the electronics module.

20. The inhalant dispensing system of claim 12, wherein the electronics module is configured to receive the data from the data module upon placement of the substance delivery component within the opening of the inhalation device.

21. A method of customizing an inhalant dispensing experience using an inhalation device comprising at least one processor, a communications device, and at least one outlet for dispensing a substance to a user in an inhalable form, the method comprising:
receiving, via the communications device, substance-identifying information for a sub stance;
determining, using the at least one processor, product preparation information associated with the substance-identifying information;
configuring, based on the product preparation information, one or more settings of the inhalation device for controlling delivery of an inhalable form of the substance, using the at least one processor; and
in response to receiving a user input, providing the substance for user inhalation via the at least one outlet.
